(19) United States
(12) Patent Application Publication
(10) Pub. No.: US 2023/0170099 A1
(43) Pub. Date: Jun. 1, 2023

(54) PHARMACEUTICAL PROCESS

(71) Applicant: MERCK PATENT GMBH, Darmstadt (DE)

(72) Inventors: Joerg WERNER, Darmstadt (DE); Dieter SCHLAPS, Wessobrunn (DE)

(73) Assignee: MERCK PATENT GMBH, DARMSTADT (DE)

(21) Appl. No.: 17/922,085

(22) PCT Filed: Apr. 29, 2021
(86) PCT No.: PCT/EP2021/061347

§ 371 Date: Oct. 28, 2022

(30) Foreign Application Priority Data
Apr. 30, 2020 (DE) 10 2020 002 607.9

(51) Int. Cl. G16H 70/40; G06F 16/903

(52) U.S. Cl. CPC G16H 70/40; G06F 16/90335

(57) ABSTRACT

The present disclosure relates to a computer-implemented method for eliminating the barriers of classical information systems and discloses a homogeneous data management system with the objective to streamline and automatize data integration for enriching pharmaceutical regulatory semantic model associated with a regulatory status of a pharmaceutical product.

The present disclosure relates to systems, methods, and computer readable media for mining regulatory information or data in pharmaceutical environment. Specifically, the present disclosure enables efficient data processing and data retrieval of a wide variety of structured or unstructured data resources for managing regulatory data relating to the development and regulatory approval of a product.

Pharmaceutical drug approval is becoming more and more difficult in markets subject to product regulation, such as the healthcare environment. Pharmaceutical, biotechnology, and medical device companies are faced with high product development costs, tough competition, and extensive regulations. The rules and procedures for obtaining regulatory review and approval often change, as do the personnel within the regulatory agencies or authorities. At the same time, companies are under enormous pressure to obtain quick regulatory approval and to keep products in compliance. Many of today’s products require regulatory approval or authorization. For instance, pharmaceutical and biotechnology companies must obtain approval from a regulatory authority, such as the U.S. Food and Drug Administration (FDA), before a new drug can be marketed. Such companies may have regulatory affairs corporate departments to manage all communications between the company and the various regulatory authorities with which it deals. The regulatory affairs department must also work with numerous other groups or divisions within the corporation, such as those responsible for quality control, research and development, and sales and marketing, to ensure that regulatory requirements are met in a coordinated fashion.

The volume of data that a regulatory affairs department must manage can be enormous. Indeed, a regulatory affairs corporate department is often responsible for numerous products subject to regulation by a host of regulatory authorities throughout the world. The amount of regulatory data for such products can grow exponentially each year as communications with those authorities continue to evolve. Moreover, companies and regulatory authorities typically require that this regulatory data be kept readily available for authority inspections and business planning.

Often, however, the regulatory data is spread over various locations throughout a company. Persons within a regulatory affairs department must often use numerous individual manual systems to track data pertaining to the products for which they are responsible. Moreover, the regulatory data is often not easily tracked, accessed, or referenced with respect to a particular product. In such environments, locating collective information pertaining to key regulatory activities is complicated and enormously time-consuming.

As data and information grow in size and complexity, knowledge management needs also have grown. Typically, larger section of data and information resides in unstructured format than in structured format in enterprises, large and small. To address the needs of data and information integration across distributed, disparate and heterogeneous data and information sources, several techniques have evolved and have been studied. In addition, several techniques describe linking unstructured data with structured data. In conventional processes of linking unstructured data with structured data, various parts of data are classified into static and dynamic parts. The aspect of identifying static and dynamic parts of data is useful to optimize various performance metrics like query time.

The explosive growth of knowledge and data is beyond the ability of traditional information management mechanisms to manage or even describe. Semantic Web technologies such as ontologies and new languages such as OWL (Web Ontology Language) and RDF (Resource Description Framework) enable the description of linked concepts such as health, medicine or engineering to be described in previously impossible detail and in a manner which is both human and machine understandable. These ontologies are typically created by teams of subject matter experts (ontologist) and are frequently publicly available.

The need for ontology alignment arises out of the need to integrate heterogeneous databases, ones developed independently and thus each having their own data vocabulary. In the Semantic Web context involving many actors providing their own ontologies, ontology matching has taken a critical place for helping heterogeneous resources to interoperate. Ontology alignment tools find classes of data that are “semantically equivalent”, for example, “Truck” and “Lorry”. The classes are not necessarily logically identical.

Also the lack of ontology assignment of the data related to the pharmaceutical regulatory processes has a risk that seamless data integration is not achieved and as a result the quality of the data is significantly reduced.

Thus, retrieving relevant structured or unstructured pharmaceutical data from disparate sources contexts is a challenge for data analysis tools. Therefore it would be advantageous to have a system and method that allows efficient retrieval of structured or unstructured data for enriching semantic models.

Accordingly, there is a need for systems, methods, that can efficiently manage regulatory data integration in the pharmaceutical industry. Moreover, there is a need for systems and methods that can manage regulatory data in the pharmaceutical industry so that it can be retrieved trackable manner, e.g., with respect to a region, a particular product or group of products, a manufacturing site, a regulation, and so forth.

The present disclosure overcomes the above identified limitations found in the prior art.

The techniques of the present disclosure may be used for mining data based on ontology matching algorithms. The enriched annotation and metadata associated with these mined data may be used for enhancing data analytics tools incorporating Artificial Intelligence (Al) and Machine Learning (ML) algorithms for analyzing the enriched sematic models.

Embodiments of the present disclosure are directed to a method, a system and a computer program of automated integration of structured and unstructured textual data sources.

The present disclosure provides methods which reliably extracts structured machine-readable contextual data from templates with diverse formats. Further, the present disclosure relates to methods and apparatuses for extracting domain specific data for enriching semantic model used in neural network and machine learning approaches for terminology enhancement. Provided are also methods and apparatuses for using controlled vocabularies for improving mining textual data relevant to pharmaceutical regulatory processes. The methods of present disclosure could be combined with existing controlled vocabularies and/or ontologies. Further, provided are computer-readable media including a program, which when executed by a computer, perform the methods of the present disclosure. The present disclosure may address the technical problems addressed above and/or other technical problems not addressed above.

The methods of the present disclosure could be used for instance building a searchable resource of Title 21 is the portion of the Code of Federal Regulations (21 CFR) that links to other regulations, guidances and regulatory processes. The methods of the present disclosure could be used alone or in combination with the known algorithms for unstructured information management for example but not limited to Unstructured Information Management Architecture (UIMA) Apache Solr NLP algorithm or the like. The use cases of the methods of the present disclosure can be for instance in extracting information related to adverse drug reactions (ADRs) from prescription drug labels in Health Leven Seven (HL7) Structured Product Labels (SPL).

Additional aspects will be set forth in part in the description which follows and, in part, will be apparent from the description, or may be learned by practice of the presented exemplary embodiments.

According to an aspect of an exemplary embodiment of the present disclosure provided is a pharmaceutical regulatory semantic model enriching system for enriching a pharmaceutical semantic model associated with a regulatory status of a pharmaceutical product comprising a data preparation unit configured to access source files, via a communication network, from a plurality of published pharmaceutical regulatory information heterogeneous data sources; a computer processing module configured to: select the source files, accessed via data preparation unit, according to a predetermined regulatory status file format, mine at least one entity from the selected source files, based on predetermined F1-measure value and according to a predetermined ontology matching algorithm, matching with user inputted queries; extract at least one dataset including ontology relevant interconnected regulatory metadata associated with the mined entity, store the said extracted dataset in a data storage unit; link the extracted dataset to one or more nodes of the pharmaceutical regulatory semantic model.

According to another exemplary embodiment of the present disclosure the pharmaceutical regulatory semantic model enriching system further comprises, the computer processing module configured to mine selected source files in multiple languages based on predetermined F1-measure value and according to a predetermined ontology matching algorithm, matching with user inputted queries.

According to another exemplary embodiment of the present disclosure the pharmaceutical regulatory semantic model enriching system further comprises a neural network device with at least two layers for mining at least one entity from the selected source files, based on a trained ontology matching algorithm, matching with user inputted queries.

According to another exemplary embodiment of the present disclosure the pharmaceutical regulatory semantic model enriching system further comprises the computer processing module configured to select data source files based on a Summary of Product Characteristics (SmPC) or a Chemistry and Manufacturing Control (CMC) file format.

According to another exemplary embodiment of the present disclosure the data preparation unit of the pharmaceutical regulatory semantic model enriching system may be configured to access source files related to Organizations Management Services (OMS) or Referential Management Services (RMS), via a communication network, from a plurality of published pharmaceutical regulatory heterogeneous data sources.

According to another exemplary embodiment of the present disclosure provided is a pharmaceutical regulatory semantic model enriching method for enriching a pharmaceutical semantic model associated with a regulatory status of a pharmaceutical product comprising: accessing source files, via a communication network, from a plurality of published pharmaceutical regulatory information heterogeneous data sources; selecting from the said accessed data sources data records based on a predetermined regulatory format; mining at least one entity from the selected source files, based on predetermined F1-measure value and according to a predetermined ontology matching algorithm, matching with user inputted queries; extracting at least one dataset including ontology relevant interconnected regulatory metadata associated with the mined entity, and storing the said extracted dataset in a data storage unit; linking the extracted dataset to one more nodes of the pharmaceutical regulatory semantic model.

According to another exemplary embodiment of the present disclosure the pharmaceutical regulatory semantic model enriching method further comprises, mining at least one entity from selected source files in multiple languages, based on predetermined F1-measure value and according to a predetermined ontology matching algorithm, matching with user inputted queries.

According to another exemplary embodiment of the present disclosure the pharmaceutical regulatory semantic model enriching method further comprises, mining at least one entity from the selected source files, based on a trained ontology matching algorithm on a neural network with at least two layers, matching with user inputted queries.

According to another exemplary embodiment of the present disclosure the pharmaceutical regulatory semantic model enriching method further comprises, selecting data source files based on a Summary of Product Characteristics (SmPC) or a Chemistry and Manufacturing Control (CMC) file format.

According to another exemplary embodiment of the present disclosure the pharmaceutical regulatory semantic model enriching method further comprises, accessing source files related to Organizations Management Services (OMS) or Referential Management Services (RMS), via a communication network, from a plurality of published pharmaceutical regulatory information heterogeneous data sources.

Although specific advantages have been enumerated above, various embodiments may include some, none, or all of the enumerated advantages.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Modifications, additions, or omissions may be made to the systems, and methods described herein without departing from the scope of the disclosure. For example, the components of the systems and methods may be integrated or separated. Moreover, the operations of the systems and methods disclosed herein may be performed by more, fewer, or other components and the methods described may include more, fewer, or other steps. Additionally, steps may be performed in any suitable order. As used in this document, “each” refers to each member of a set or each member of a subset of a set.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a conceptual diagram illustrating a pharmaceutical regulatory semantic model enriching system (SMES) according to an exemplary embodiment;

FIG. 2 is a diagram for describing a computational steps performed by the pharmaceutical regulatory semantic model enriching system (SMES) according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The terms “according to some exemplary embodiments” or “according to an exemplary embodiment” used throughout the specification do not necessarily indicate the same exemplary embodiment.

Some exemplary embodiments of the present disclosure may be represented by functional block configurations and various processing operations. Some or all of these functional blocks may be implemented using various numbers of hardware and/or software components that perform particular functions. For example, the functional blocks of the present disclosure may be implemented using one or more microprocessors or circuits for a given function. Also, for example, the functional blocks of the present disclosure may be implemented in various programming or scripting languages. The functional blocks may be implemented with algorithms running on one or more processors. The present disclosure may also employ conventional techniques for electronic configuration, signal processing, and/or data processing. The terms “mechanism”, “element”, “unit” and “configuration” may be used in a broad sense and are not limited to mechanical and physical configurations, and may be implemented in hardware, firmware, software, and/or a combination thereof.

Also, connection lines or connection members between the components illustrated in the drawings are merely illustrative of functional connections and/or physical or circuit connections. In actual devices, connections between the components may be represented by various functional connections, physical connections, or circuit connections that may be replaced or added.

Meanwhile, with respect to the terms used herein, template may refer to any executable or non-executable file format with different file extensions. Template may also refer to any image representation of a physical or a virtual document like webpages or scanned images or any other virtual entity from which it is possible to obtain digitalized information regarding the chemical structure(s). The image representation of the template may comprise a complete of a partial section(s) of the physical or the virtual document. The template may also comprise of standard exchange file formats compatible with the regulatory guidelines like, but not limited to, Summary of Product Characteristics (SmPC) or Chemistry, Manufacturing, and Controls (CMC) Regulatory Affairs (RA) or the like.

In addition, an ontology may refer to a vocabulary and a specification of the meaning of terms used in the vocabulary describing pharmaceutical regulatory processes. For example, but not limited to, the ontologies can comprise the descriptors used for describing the information of in the SmPC or Chemistry, Manufacturing and Controls (CMC) Module 3. This may include for example, name of the medicinal product, qualitative and quantitative composition, pharmaceutical form, clinical particulars for example posology and methods of administration, contraindications, overdose, undesirable effects or the like, pharmacological properties for example pharmacodynamic or pharmacokinetic properties, or pharmaceutical particulars for example, shelf life, nature and contents of container or the like.

In addition, heterogeneous data sources may refer but not limited to data sources comprising both of structured, semi-structured and unstructured data sources Structured data is data that adheres to a pre-defined data model and is therefore straightforward to analyze. Structured data conforms to a tabular format with relationship between the different rows and columns. Common examples of structured data are Excel files or SQL databases. Each of these have structured rows and columns that can be sorted. Unstructured data is information that either does not have a predefined data model or is not organized in a pre-defined manner. Unstructured information is typically text-heavy, but may contain data such as dates, numbers, and facts as well. This results in irregularities and ambiguities that make it difficult to understand using traditional programs as compared to data stored in structured databases. Common examples of unstructured data include audio, video files or No-SQL databases. Semi-structured data is a form of structured data that does not conform with the formal structure of data models associated with relational databases or other forms of data tables, but nonetheless contain tags or other markers to separate semantic elements and enforce hierarchies of records and fields within the data. Metadata is a data about data. It is not a separate data structure and provides additional information about a specific set of data of any category as listed above.

In addition, mine may refer to analyzing large amounts of data in order to discover patterns or selecting data from a large amounts of data based on parameter values or attributes. It may also be process of trying to get more refined data sets out of a large data set.

In addition, the term “meaning” is intended to refer to the semantic interpretation of a particular ontology term, content field name, or the like. The term meaning therefore encompasses the intended meaning of the ontology term or content field, for example to account for issues such as homonyms, synonyms, meronyms, or the like, as will be described in more detail below.

In addition term matching may refer to ontology matching. In technical terms it is semantic mapping between two ontologies for example user inputted queries and mined entities using an ontology matching algorithm. The term entity may refer to semantically mapped ontology based on the inputted query of the user.

In addition, term link may refer to creation of links between sematic model and metadata associated with mined entities. It creates a linked data paradigm allowing the reuse of existing knowledge. Linked Data standards may be applied to metadata for example, Resource Description Framework (RDF) for metadata. Thus the linked data by leveraging existing vocabularies can be used for enhancing the existing semantic model.

For the purpose of illustration throughout the following description, the term “source” is used to refer to a data store, such as a database or file from which data is being extracted, whilst the term “target” is used to refer to a data store, such as a database or file into which data is being stored. These terms are for the purpose of illustration only, for example to distinguish between possible sources and targets, and are not intended to be limiting. The term “content instance” refers to an individual piece of content that is being extracted from a source and/or transferred to a target and is also not intended to be limiting. For example the term content instance could refer to a database record having values stored in a number of different database fields, or a set of related database records, or could alternatively refer to a single value stored within a single field.

In addition, domain can refer to any hierarchical categorization in the guidelines related to the regulatory processes for example, but not limited to, Summary of Product Characteristics (SmPC) or Chemistry, Manufacturing, and Controls (CMC) Regulatory Affairs (RA) or the like.

In addition, rule set, may refer to matching ontologies by finding correspondences between semantically related entities of ontologies. This reduces the semantic gap between different overlapping representations of the same domain. These correspondences can be used for various tasks, such as ontology merging, query answering, or data translation. Thus, matching ontologies enables the knowledge and data expressed with respect to the matched ontologies to interoperate. The methods of the present disclosure may be used with any known ontology matching algorithms for example, but not limited to, formal or informal resource-based, string-based, language-based, constraint-based, taxonomy-based, draft-based instance-based or model-based or the like.

In addition an artificial neural network (ANN) may refer to a collection of fully or partially connected units comprising information to convert input data into output data.

In addition, a Machine Learning (ML) may refer to ML-based ontology alignment system using a classifier using techniques for example, but not limited to, Support Vector Machine (SVM), K-Nearest Neighbors (KNN), Decision Tree (DT), AdaBoost or the like.

In addition, metric measure may refer to a metric for evaluating ontology-based information extraction. The present disclosure can be combined with different types of metrics for example, but not limited to Cost-based Evaluation Metric, Learning Accuracy measures measuring how well the ontology is populated, Augmented Precision and Recall metric or F1 measure which uses Precision, Recall metrics. Where precision measures the number of correctly identified items as a percentage of the number of items identified and Recall measures the number of correctly identified items as a percentage of the total number of correct items.

Also, structured data refers to data with any kind of information which is added as meta data to the original data in order to group parts of the original data, facilitating the automatic downstream processing of the resulting information.

Hereinafter, exemplary embodiments of the present disclosure will be described in detail with reference to the accompanying drawings.

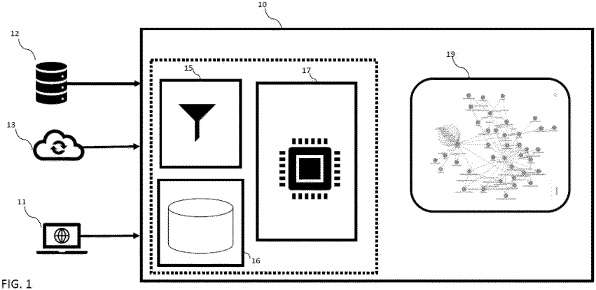

An example of the process for enriching a pharmaceutical regulatory semantic model from external databases such as published pharmaceutical regulatory databases or the like, will now be described with reference to FIG. 1.

FIG. 1 depicts an exemplary process illustrating an example of a pharmaceutical regulatory semantic model enriching system (SMES) 10. The SMES 10 includes a network interface (not shown), a Data Preparation Unit (DP) 15, a Data Storage Unit (DI) 16, a computer processing module 17, a Data Curator and Integrator Unit (DC) (not shown), a user Interface (not shown), and a semantic model for regulatory process 19.

The pharmaceutical regulatory semantic model enriching system (SMES) 10 is connected via the network interface 14 with external data sources like external databases 12, cloud-based services 13, web resources 11.

The SMES 10 is controlled through an intuitive user interface (Ul) (not shown in the FIG. 1) by which the user composes and submits queries; reviews the information found; selects report preferences; and outputs (e.g.; prints) reports. Users are identified and their access is authenticated through a security system upon requesting access to the SMES 10 via assigned user passwords and identifiers. The identifiers define the user’s level of access and the types of information they have permission to access. For example, a user may only be interested in accessing regulatory information relating to medical devices. As such, other regulatory information categories (i.e., pharmaceutical or environmental hazards) would not be accessible.

The SMES 10 may access source files from a plurality of heterogeneous information sources, each of which may have different information types (e.g.; different files, different records with each file, different fields within each record, etc.). Some information types are extracted from public websites 11, where this information may reside within the text of a web page or in a downloadable file. For example, the European Medicine Agency (EMA) publishes information on human or veterinary medicines (pharmaceutical products) at various stages of their lifecycles, from early development through initial evaluation to post-authorization changes, safety reviews and withdrawals of authorization. Also by way of example, adverse event reports for medical devices are typically contained in a downloadable file that can be imported into a database and available from MedDRA - the Medical Dictionary for Regulatory Activities.

Each accessed data source has its own characteristics and style for presenting data. Thus, the data from each source has a defined set of rules and a regimen for conversion within the Data Preparation Unit DP 15. Each information type in the accessed data records can be converted into a consistent digital format suitable for importing into an electronic database. For example, data retrieved may be in a portable data format (.PDF) or in a tab-separated text format. A table published on a web page is extracted, broken down into specified data fields, and converted into a spreadsheet or into tab-separated text. Appropriate conversion of the accessed data records is completed prior to the data extraction step.

Data corrections also are made by Data Preparation Unit DP 15 for data inconsistencies to allow consolidation and integration of data from multiple sources. Errors can exist in data sets obtained from an information source. For example, the data listing for clinical investigators of drug clinical trials can include multiple listings that begin with a sequence of “YYY”. If this data was not corrected, searches for “Manuel Schmidt” would not recognize a record for “Manuel YYYSchmidt”. A means for identifying such errors and correcting them, such as one or more predetermined filters, can be provided by software and/or hardware. As new discrepancies are discovered, the system and method can add, alter, or delete one or more predetermined filters so as to identify and correct discrepancies as they are identified.

Over time, the information sources may change the way that the information is collected and/or reported. For example, information sources are increasingly converting their frequently used information (for example, adverse event reports or establishment registrations) into a searchable format via a web interface. The SMES 10 includes internal checks that detect changes that occur in order to appropriately adjust the data access frequency.

Inconsistency in terminology is likely across heterogeneous information sources (e.g.; disparate data sources), which may be due to each data source having been created with a specific use in mind that differed from that of other data sources. These data must then be normalized before data curation and integration 18. As regulatory requirements change, an entire scheme of information may change. The SMES 10 detects and allows compensation for these changes.

The computer processing module 17, based on the user’s input or a list of inputted queries, mine entities by performing an ontology matching on the accessed data sources. This returns may return ontology matched data records from the accessed data sources. Alternatively, also data sets from the matched data records of the accessed data sources can also be extracted by the pharmaceutical regulatory semantic model enriching system (SMES) 10 of the present disclosure.

The computer processing module 17 according to the present example enables semantic matching by considering relationships between elements of the accessed data records and its metadata elements to enhance scope of the ontology matching.

The computer processing module 17 may attempt to extend a scope of the search results to regulatory status documents such as spreadsheet documents that contains tables, charts, reports, diagrams, filtered charts/tables, and similar elements. Some of these elements may be generated by an application other than the spreadsheet application associated with the spreadsheet document and embedded into the spreadsheet document statically or dynamically (i.e. element data residing at an external source). Example spreadsheet documents in the accessed data sources may include textual report, table, chart, and video data (presentation). Textual report includes links to the individual non-textual elements. Furthermore, table and chart may be associated (e.g. part of the data in table may be displayed in chart). Other relationships are also possible.

The computer processing module 17 may extract metadata that contains the details of the regulatory status related information. For example, a spreadsheet document in the accessed data records may include multiple sheets filtering tables. Each filtering table may include a variety of filters. The spreadsheet document may further include diagrams and/or charts based on data that is stored in the spreadsheet document and/or stored at an external resource (e.g. another spreadsheet document, a data store, etc.). The charts and/or diagrams may be generated based on filtering the data according to one or more of the filters in the filtering table. Thus, the elements in the spreadsheet document may not reflect the entire extent of available data. Moreover, relationships between the elements (e.g. between the tables and charts, video data and tables, etc.) may be useful to a user in determining the importance or relevance of retrieved data and driving a search client user interface and result display dynamically.

Because the data in the spreadsheet document may be limited (e.g. filtered from the available data at the external data source), computer processing module 17 may retrieve additional information from the data source to enrich the search results. For example, additional dimension members beside the applied filter members may be retrieved from the data at the data source. Dimensions, hierarchies, and measure information of stored data may also be retrieved. Thus, detailed metadata and dataset may be extracted in a structural and meaningful manner and used to scope the search results into regulatory status related documents and dynamically drive variations in result content display of a rendering application.

Whilst this example is specific to select data records from a relational database, it will be appreciated that similar concepts can be applied to other data structure or unstructured data sources, and that this example is for the purpose of illustration only and is not intended to be limiting.

The extracted data records and/or datasets can be stored in the local Data Storage Unit 16 for further processing and subsequent usage.

The output of the computer processing module 17 is inputted to the Data Curator and Integrator Unit (DC). DC performs a quality check on the extracted data records or datasets both including the associated metadata and semantically links the extracted information to one or more nodes of the pharmaceutical regulatory semantic model. Thus, the pharmaceutical regulatory semantic model is enriched.

An example of extraction based on F-measure value performed by the computer processing module 17 using ontology matching algorithm will now be described.

An F-score is a measure of algorithmic fidelity and may be computed based on ontology comparison algorithm precision and recall. Precision is a measure of exactness or fidelity, whereas recall is a measure of completeness. Precision and recall may be based on true positives (tp), true negatives (tn), false positives (fp), and false negatives (fn) of the concept string associations. Precision may be based on the following equation:

$$\text{precision=tp/(tp+fp)}$$

Recall may be based on the following equation:

$$\text{recall=tp/(tp+fn)}$$

In the above embodiment, the closer the F1-score value is to 1.0, the higher the degrees of both precision and recall. The following equation may be used to compute F1-score value:

$$\text{F1-score value=2*(precision*recall)/(precision+recall)} \text{.}$$

The pharmaceutical regulatory semantic model enriching system (SMES) performs mining using controlled vocabularies and entities in the source file are mined based on F1 score between 0.95 and 1.

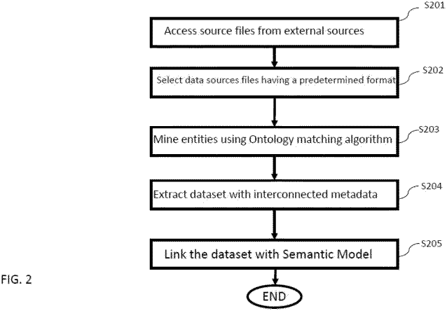

FIG. 2 depicts an exemplary methods steps for enriching a pharmaceutical regulatory semantic model associated with a regulatory status of a pharmaceutical product.

In step S201 the data preparation unit 15 accesses source files, via a communication network, from a plurality of published pharmaceutical regulatory information heterogeneous data sources. Data can be accessed from a variety of sources like external databases 12, cloud-based services 13, web resources 11. The data can be accessed through a database connection which allows the pharmaceutical regulatory semantic model enriching system (SMES) to talk to database server software. An application driver may be used with SMES wherein the information needed to connect to a database or cloud-services or the like is included in the SMES which prompts the user to authenticate before establishing the connection. Alternatively, Instance merge modules may be used for creating an instance environment which serves to establish the connection. The SMES may include sockets or the like for accessing data servers over the web.

In Step S202, the computer processing module 17, select source files according to a predetermined regulatory status file format. This may be executed by creating filters on a data source, thereby reducing the amount of data to be selected from the available in the data sources. For example Javascript/jQuery Grid with frameworks like Angular and ReactJS can be used to select source files conforming to a predetermined regulatory status file format.

In step S203, the ontological matching algorithm mines the entities matching with user inputted queries based on a predetermined F1-measure value. Generally F1-measure value is chosen to be as near to 1 as possible. Ontology matching algorithms for example, but not limited to, formal or informal resource-based, string-based, language-based, constraint-based, taxonomy-based, draft-based instance-based or model-based or the like may be used.

In step S204, the computer processing module 17 extracts data set including metadata associated with the mined entity. This may be implemented using web scrapping tools or techniques likes Document Parsing or Tokenization. Alternatively, techniques like Named Entity Recognition may be used to identify important names like drug content, dosage, disease etc from text. In Step 204 the SMES may use either training based methods/gazetteer and grammar based for named entity recognition. Also, sequence labeling methods like conditional random fields or Hidden Markov models may be used for training based approach. Semantic Parsing may be used to analyze different syntax and semantic aspects in text and connect different words present in unstructured data. It will be evident to the person skilled in the art that this step may also be implemented with standalone data extraction tools in combination with SMES 10.

In Step S205a (not shown), the extracted data set may be stored locally for reuse. Alternatively, the extracted data set may be directly used for linking the data set including metadata for enriching a pharmaceutical regulatory semantic model associated with a regulatory status of a pharmaceutical product.

In Step S205 the system according the present disclosure links the extracted data set including meta data for enriching a pharmaceutical regulatory semantic model associated with a regulatory status of a pharmaceutical product. It may be implemented by creating links between sematic model and metadata associated with mined entities. Linked Data standards may be applied to metadata for example, Resource Description Framework (RDF) for metadata. Links may be established using a HTML anchors.

An example of the pharmaceutical regulatory semantic model enriching system (SMES) according to the present disclosure could be in language aware ontology matching. Language aware or multilingual matching as a type of ontology matching where a pharmaceutical regulatory semantic model enriching system (SMES) can match ontologies expressed in multiple languages. The pharmaceutical regulatory semantic model enriching system according to this example of the present disclosure comprises an extensible multilingual knowledge base as principal source of background knowledge and a multilingual label processor, extensible to new languages. The background knowledge is a knowledge base containing lexical databases (i.e., wordnet) for each language supported, a language-independent ontology of concepts serving as an interlingua. Label processing consists of a language aware label parsing step. Label parsing is a multilingual natural language processing task optimized to the language of lightweight ontology labels and is extensible by language-specific NLP components. Label parsing consists of the following sub steps: (a) language detection that makes the language of each input tree explicit, and computation of formula structure that parses the label using syntactic NLP techniques partly generalized and partly adapted to each language supported, computation of atomic concepts that formalizes meaningful words in the label as language-independent concepts.

Thus, the multilingual source files can be mined and can serve to enrich the pharmaceutical regulatory semantic model.

According to another example of the present disclosure the pharmaceutical regulatory semantic model enriching system (SMES) may include a supervised or non-supervised machine learning device.

The machine learning device operate in two phases (i) the learning or training phase and (ii) the classification or matching phase. During the learning phase the training that for the learning process is created, for example, by manually matching two ontologies, so that the system learns a matcher (trained ontology matching algorithm) from this data. During the classification of matching phase the learnt ontology matching algorithm is used for mining the relevant metadata from the external source files. The accuracy of the mined dataset is feedback to the system for further improvement.

Thus, the semantic model is enriched.

Also, the aforementioned examples may be embodied in the form of a recording medium including instructions executable by a computer, such as a program module, executed by a computer. The computer-readable medium may be any recording medium that may be accessed by a computer and may include volatile and non-volatile media and removable and non-removable media. The computer-readable medium may include a non-transitory computer-readable medium that stores one or more instructions that, when executed by one or more processors, cause the one or more processors to perform operations associated with exemplary embodiments described herein. Also, the computer-readable medium may include computer storage media and communication media. The computer storage media include volatile and non-volatile and removable and non-removable media implemented using any method or technology to store information such as computer-readable instructions, data structures, program modules, or other data. The communication media include computer-readable instructions, data structures, program modules, or other data in a modulated data signal, or other transport mechanisms and include any delivery media.

In addition, throughout the specification, the term “system” may be a hardware component such as a microprocessor or a circuit and/or a software component executed by the hardware component such as a FGPA.

The above description of the present disclosure is provided for the purpose of illustration, and it should be understood by those skilled in the art that various changes and modifications may be made without changing technical conception and essential features of the present disclosure. Thus, it is clear that the above-described illustrative exemplary embodiments are illustrative in all aspects and do not limit the present disclosure. For example, each component described to be of a single type may be implemented in a distributed manner. Likewise, components described to be distributed may be implemented in a combined manner.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

1. A pharmaceutical regulatory pharmaceutical regulatory semantic model enriching system for enriching a semantic model associated with a regulatory status of a pharmaceutical product comprising:
a data preparation unit configured to access source files, via a communication network, from a plurality of published pharmaceutical regulatory information heterogeneous data sources;
a computer processing module configured to:
select the source files, accessed via data preparation unit, according to a predetermined regulatory status file format;
mine at least one entity from the selected source files, based on predetermined F1-measure value and according to a predetermined ontology matching algorithm, matching with user inputted queries;
extract at least one dataset including ontology relevant interconnected regulatory metadata with the mined entity,
store the said extracted dataset in a data storage unit;
link the extracted dataset to one more nodes of the pharmaceutical regulatory semantic model.

2. The system according to claim 1 further comprising, the computer processing module configured to mine selected source files in multiple languages based on predetermined F1-measure value and according to a predetermined ontology matching algorithm, matching with user inputted queries.

3. The system according to claim 1 further comprising, a neural network device with at least two layers for mining at least one entity from the selected source files, based on a trained ontology matching algorithm, matching with user inputted queries.

4. The system according to claim 1, further comprising the computer processing module configured to select data source files based on a Summary of Product Characteristics (SmPC) or a Chemistry and Manufacturing Control (CMC) file format.

5. The system according to claim 1 , wherein the data preparation unit is configured to access source files related to Organisations Management Services (OMS) or Referentials Management Services (RMS), via a communication network, from a plurality of published pharmaceutical regulatory heterogeneous data sources.

6. A pharmaceutical regulatory semantic model enriching method for enriching a semantic model associated with a regulatory status of a pharmaceutical product comprising:
accessing source files, via a communication network, from a plurality of published pharmaceutical regulatory information heterogeneous data sources;
selecting from the said accessed data sources data records based on a predetermined regulatory format;
mining at least one entity from the selected source files, based on predetermined F1-measure value and according to a predetermined ontology matching algorithm, matching with user inputted queries;
extracting at least one dataset including ontology relevant interconnected regulatory metadata with the mined entity,
storing the said extracted dataset in a data storage unit;
linking the extracted dataset to one more nodes of the pharmaceutical regulatory semantic model.

7. The method according to claim 6 further comprising, mining at least one entity from selected source files in multiple languages, based on predetermined F1-measure value and according to a predetermined ontology matching algorithm, matching with user inputted queries.

8. The method according to claim 6, further comprising, mining at least one entity from the selected source files, based on a trained ontology matching algorithm on a neural network with at least two layers, matching with user inputted queries.

9. The method according to claim 6, further comprising, selecting data source files based on a Summary of Product Characteristics (SmPC) or a Chemistry and Manufacturing Control (CMC) file format.

10. The method according to claim 6, further comprising, accessing source files related to Organisations Management Services (OMS) or Referentials Management Services (RMS), via a communication network, from a plurality of published pharmaceutical regulatory information heterogeneous data sources.

11. A computer-readable medium comprising instructions which, when executed by a computer, cause the computer to carry out the steps of claim 6.

12. A computer program comprising instructions which, when the program is executed by a computer, cause the computer to carry out the steps of claim 6.
